(19) United States
(12) Patent Application Publication
(10) Pub. No.: US 2013/0344550 A1
(43) Pub. Date: Dec. 26, 2013

(54) METHODS OF BIOPLASTIC PRODUCTION

(71) Applicants: Charles Miller, North Logan, UT (US); Asif Rahman, Logan, UT (US); Ronald Sims, Logan, UT (US); Ashik Sathish, North Logan, UT (US); Renil Anthony, Salt Lake City, UT (US)

(72) Inventors: Charles Miller, North Logan, UT (US); Asif Rahman, Logan, UT (US); Ronald Sims, Logan, UT (US); Ashik Sathish, North Logan, UT (US); Renil Anthony, Salt Lake City, UT (US)

(73) Assignee: Utah State University, North Logan, UT (US)

(21) Appl. No.: 13/914,461

(22) Filed: Jun. 10, 2013

Related U.S. Application Data
(60) Provisional application No. 61/657,649, filed on Jun. 8, 2012.

(51) Int. Cl. C12P 7/62

(52) U.S. Cl. CPC C12P 7/625
USPC 435/135; 435/253.6

(57) ABSTRACT

A method of producing bioplastics from algae, the method including processing algae to yield an aqueous phase containing glycerol, and fermenting the aqueous phase with a bioplastic-producing bacteria to yield bioplastics.

### CROSS-REFERENCE TO RELATED APPLICATION

This application claims priority to U.S. Provisional Application No. 61/657,649, filed on Jun. 8, 2012, the entirety of which is hereby incorporated by reference.

### GOVERNMENT SPONSORED RESEARCH

The inventions described herein were made at least in part with government support under contract DE-EE0003114 awarded by the United States Department of Energy. The government has certain rights in the inventions.

### TECHNICAL FIELD

The present disclosure relates to the production of bioproducts from biomass, more particularly, it relates to methods and materials for producing bioplastics from algal biomass.

### BACKGROUND

The production of bioproducts from various biological feedstocks has been explored in an effort to enable sources of renewable and biodegradable plastics. However, improved and additional methods for processing biomass into bioplastics are needed for commercial viability and/or feasibility to be established. Biodegradable bioplastic in the form of polyhydroxyalkanoates (PHA) (or more specifically polyhydroxybutyrates (PHB)) may be produced from genetically engineered Escherichia coli grown on waste carbon sources. See Koller et al., Microbial PHA production from waste raw materials, Plastics from Bacteria. Edited by Chen G Q. Springer 2010.

### SUMMARY

The present disclosure in aspects and embodiments addresses these various needs and problems by providing methods, compositions, reagents, and kits for producing bioplastics from algae, the method including processing algae to yield an aqueous phase containing glycerol, and fermenting the aqueous phase with a bioplastic-producing bacteria to yield bioplastics.

### BRIEF DESCRIPTION OF THE DRAWINGS

FIG. 1 illustrates a CFU/mL for various exemplary samples.

FIG. 2 is an NMR for a product produced according to an exemplary plastic production method.

FIG. 3 is an NMR for a product produced according to an exemplary plastic production method.

FIG. 4 is an NMR for a product produced according to an exemplary plastic production method.

FIG. 5 is an NMR for a product produced according to an exemplary plastic production method.

FIG. 6 is an NMR for a control product.

FIG. 7 is an NMR for a product produced according to an exemplary plastic production method.

FIG. 8 is an NMR for a product produced according to an exemplary plastic production method.

FIG. 9 is an NMR for a product produced according to an exemplary plastic production method.

FIG. 10 illustrates OD600 v. time for different concentrations of glycerol in M9 media.

FIG. 11 is an NMR for exemplary PHB secreting strains.

### DETAILED DESCRIPTION

The present disclosure covers apparatuses and associated methods for producing bioplastics from algal biomass. In the following description, numerous specific details are provided for a thorough understanding of specific preferred embodiments. However, those skilled in the art will recognize that embodiments can be practiced without one or more of the specific details, or with other methods, components, materials, etc. In some cases, well-known structures, materials, or operations are not shown or described in detail in order to avoid obscuring aspects of the preferred embodiments. Furthermore, the described features, structures, or characteristics may be combined in any suitable manner in a variety of alternative embodiments. Thus, the following more detailed description of the embodiments of the present invention, as illustrated in some aspects in the drawings, is not intended to limit the scope of the invention, but is merely representative of the various embodiments of the invention.

In this specification and the claims that follow, singular forms such as “a,” “an,” and “the” include plural forms unless the content clearly dictates otherwise. All ranges disclosed herein include, unless specifically indicated, all endpoints and intermediate values. In addition, “optional” or “optionally” refer, for example, to instances in which subsequently described circumstance may or may not occur, and include instances in which the circumstance occurs and instances in which the circumstance does not occur. The terms “one or more” and “at least one” refer, for example, to instances in which one of the subsequently described circumstances occurs, and to instances in which more than one of the subsequently described circumstances occurs.

The present disclosure covers methods, compositions, reagents, and kits for making bioplastics from algal biomass. The methods described herein may be accomplished in batch processes or continuous processes.

I. Feedstocks and Flocculation

A. Feedstocks

As a feedstock, any suitable algae may be used. In embodiments, algae that produce high concentrations of polysaccharides may be preferred. In many embodiments, algae produced in wastewater may be used. The algae may be lyophilized, dried, in a slurry, or in a paste (with for example 10-15% solid content).

Any suitable algae harvesting method may be used alone or in combination with one another. For example, the algae may be harvested using a rotating bioreactor, as described in U.S. patent application Ser. No. 13/040,364 (herein incorporated by reference in its entirety). In addition to or independent from, the algae may be harvested using inorganic or organic coagulants/flocculants as described in U.S. Provisional Patent Application 61/552,604 (herein incorporated by reference in its entirety).

When organic coagulants/flocculants are used, the feedstock will include both the algae and the organic coagulant/flocculant.

B. Flocculation

In embodiments, organic coagulants and flocculants may be employed to effectively harvest algae without negatively affecting the various bio-products that may be later derived from algae. Exemplary bio-products of algae include bio-plastics, biodiesel, bio-solvents, and numerous other products.

(1) Starch

Starch is an abundant natural polymer available from sources such as potato, corn, rice, tapioca, etc. Irrespective of the source, starch is primarily comprised of amylose (20-30% wt) and amylopectin (70-80% wt), which are illustrated below:

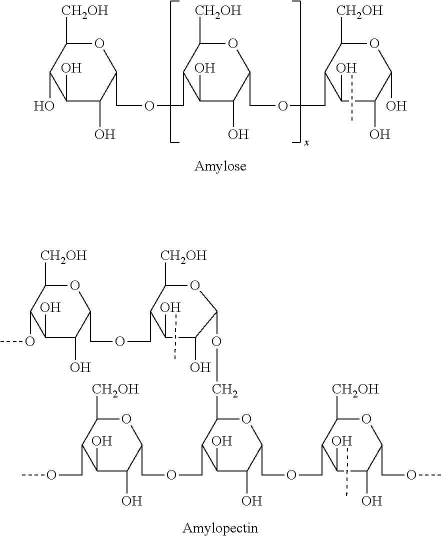

In some embodiments, the starch source may be what would otherwise be considered a waste product, such as waste starch derived from potato, or other vegetable, processing.

The starch may be modified to have cationic groups, such as amine, ammonium, phosphonium, or imines. By modifying the starch with cationic groups, the starch may then serve as an organic coagulant and flocculant for algae harvesting.

(2) Starch Modification

The starch may be modified by any suitable method. In some embodiments, the starch is modified by initiating free radicals on the starch backbone and grafting a quaternary ammonium moiety on to it, as set forth in the following reaction scheme:

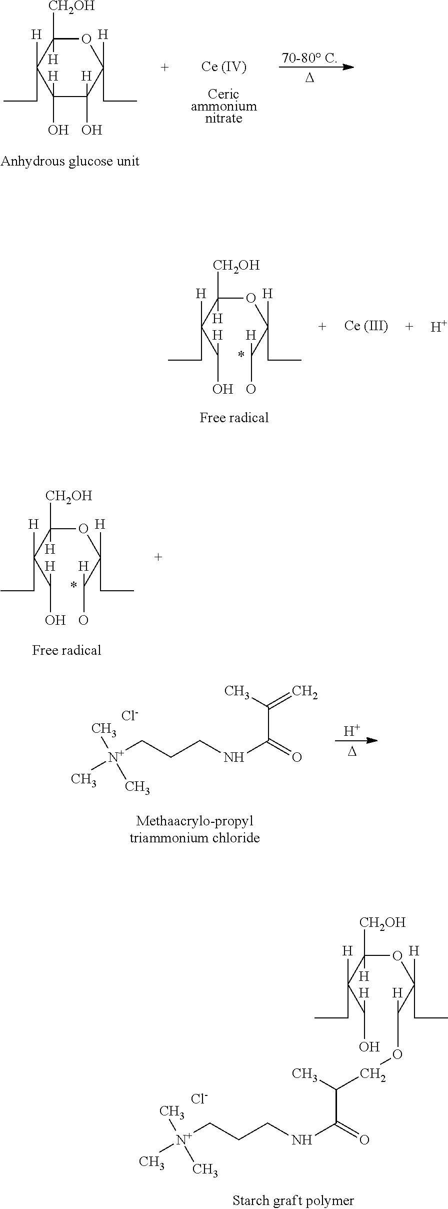

The free radical generation and quaternary ammonium can be achieved through other chemicals and reagents. For example, for the free radical generation ferrous ion-peroxide or potassium persulfate/sodium thiosulfate redox system can be used and [2-(Methacryloyloxy)-ethyl]-trimethylammoniumchloride (TMAEMA) or [3-(Methacryloylamino)-propyl]-trimethylammoniumchloride (MAPTAC) or Diallyldimethylammoniumchloride (DADMAC) can be used as quaternary ammonium.

To begin with, free radicals on the starch backbone (e.g. corn or potato) can be generated by addition of ceric ammonium nitrate to a gelatinized starch mixture at 60-90° C. for 15-60 minutes. After generating free radicals, [3-(Methacryloylamino)-propyl]-trimethylammoniumchloride (MAPTAC) is added and the mixture is made acidic to pH 2-4 by the addition of nitric acid. The mixture is heated for 2-6 hours at 60-90° C. after which it is allowed to slowly cool to room temperature.

This modified starch may be separated from the solution by precipitation with, for example, ethanol. The solution may be centrifuged, or otherwise subjected to a solid-liquid separation technique, to collect the precipitate and the supernatant may then be discarded. The precipitate may be washed with a suitable washing agent, such as ethanol in a soxhlet apparatus with a reflux time which may include up to 20 hours, such as about 5 to 15 hours, or about 12 hours to clean the starch of any unreacted reagents and catalyst. The modified starch may then be dried of the washing agent, optionally pulverized, and stored at room temperature until further use.

After modified starch preparation, the zeta potential may be measured to examine the potency of the modified starch as a potential coagulant and flocculant. Zeta potential is the measure of charge present on a colloidal particle surface. For the modified starch to show cationization, the zeta potential should be greater than 0. Minimum zeta potential above about +1 mV is necessary for the feasibility of starch as a coagulant/flocculant for algae separation and harvesting. Suitable zeta potentials for the modified starch as a coagulant/flocculant may include, for example, from about +5 to about +20 mV in a pH of about 5.0 to about 10.0.

Degree of substitution (DS) relates to the number of hydroxyl groups (maximum 3) that are substituted by quaternary ammonium. In embodiments, the higher the degree of substitution, the greater would be the neutralizing capability of a modified starch resulting in efficient separation with minimal dosage. Suitable DS values may include, for example, from about 0.82 to about 1.34.

(3) Precipitate Formation

The CAS, or modified starch, may be mixed with an aqueous solution containing algae to be harvested. Suitable ratios include, for example, from about 0.5:1.0 to 3.0:1.0 starch:algae. Upon addition of the modified starch, the solution may be optionally flash mixed to facilitate uniform mixing of the modified starch in the suspension for charge neutralization and to avoid lump formation. Flash mixing may be followed by slow mixing to facilitate bridging (particle interaction between algae and starch) of the neutralized algae particles and also to help in residual charge neutralization not achieved by flash mixing. The mixing may be then stopped and the flocs are allowed to sediment for a period of time. Precipitate formation may be performed in a suitable reactor equipped with optional stirrers and/or convection properties.

(4) Slurry Formation

After identification and/or harvesting of a feedstock source or sources, the algae may be formed into a slurry, for example, by adding water, adding dried or lyophilized algae, or by partially drying, so that it has a solid content of about 1-40%, such as about 4-25%, about 5-15%, about 7-12%, or about 10%.

II. Algal Biomass Pre-Processing

In some embodiments, the feedstock may optionally be pre-processed into a processed biomass prior to bioplastic production, as described, inter alia, in U.S. Provisional Patent Application 61/551,049, the entirety of which is herein incorporated by reference. This pre-processing, also referred to as Wet Lipid Extraction Process (“WLEP”), to yield processed biomass may include cell lysis, and solid/liquid separation as described, for example, below. Pre-processing the feedstock can lead to increased bioplastics production during fermentation.

A. Algal Cell Lysis

The algal cells in the feedstock may be optionally lysed by any suitable method, including, but not limited to acid and/or base hydrolysis (described below). Other methods may include mechanical lysing, such as smashing, shearing, crushing, and grinding; sonication, freezing and thawing, heating, the addition of enzymes or chemically lysing agents, or any combination of the above.

In some embodiments, the algal cells may be lysed by acid hydrolysis followed by an optional base hydrolysis.

(1) Acid Hydrolysis

To degrade the algal cells (or other cells present), to bring cellular components into solution, and to break down complex components, such as polysaccharides to their respective monosaccharide components as well as lipids to free fatty acids, a slurry of water and feedstock, as described above, may be optionally heated and hydrolyzed with at least one acidic hydrolyzing agent.

Complex carbohydrates may include, but are not limited to, starch, cellulose, and xylan. The degradation of these complex polysaccharides from the acid hydrolysis will yield oligosaccharides or monosaccharides that can be readily used for bioplastics production. These complex lipids may include, for example, triacylglycerols (TAGs), phospholipids, etc. In addition to degrading algal cells and complex lipids, the acidic environment created by addition of the hydrolyzing agent removes the magnesium from the chlorophyll molecules.

When heated, the slurry may reach temperatures of from about 1-200° C., such as about 20-100° C., about 50-95° C., or about 90° C. When temperatures above 100° C., or the boiling point of the solution are used, an apparatus capable of withstanding pressures above atmospheric pressure may be employed. In some embodiments, depending on the type of algae, the type and concentration of acid used for hydrolysis, the outside temperature conditions, the permissible reaction time, and the conditions of the slurry, heating may be omitted. Heating may occur prior to, during, or after addition of a hydrolyzing agent.

In addition, the slurry may be optionally mixed either continuously or intermittently. Alternatively, a hydrolysis reaction vessel may be configured to mix the slurry by convection as the mixture is heated.

Acid hydrolysis may be permitted to take place for a suitable period of time depending on the temperature of the slurry and the concentration of the hydrolyzing agent. For example, the reaction may take place for up to 72 hours, such as from about 12-24 hours. If the slurry is heated, then hydrolysis may occur at a faster rate, such as from about 15-120 minutes, 30-90 minutes, or about 30 minutes.

Hydrolysis of the algal cells may be achieved by adding to the slurry a hydrolyzing agent, such as an acid. Any suitable hydrolyzing agent, or combination of agents, capable of lysing the cells and breaking down complex carbohydrates and lipids may be used. Exemplary hydrolyzing acids may include strong acids, mineral acids, or organic acids, such as sulfuric, hydrochloric, phosphoric, or nitric acid. These acids are all capable of accomplishing the goals stated above. When using an acid, the pH of the slurry should be less than 7, such as from about 1-6, about 1.5-4, or about 2-2.5.

In addition to strong acids this digestion may also be accomplished using enzymes alone or in combination with acids that can break down plant material. However, any such enzymes or enzyme/acid combinations would also be capable of breaking down the complex polysaccharides to their respective oligosaccharides or monosaccharides as well as complex lipids to free fatty acids.

In some embodiments, the acid or enzymes, or a combination thereof, may be mixed with water to form a hydrolyzing solution. However, in other embodiments, the hydrolyzing agent may be directly added to the slurry.

(2) Base Hydrolysis

After the initial acidic hydrolysis, a secondary base hydrolysis may be performed to digest and break down any remaining whole algae cells; hydrolyze any remaining complex polysaccharides and lipids and bring those polysaccharides and lipids into solution; convert all free fatty acids to their salt form, or soaps; and to break chlorophyll molecules apart.

In this secondary hydrolysis, the biomass in the slurry may be mixed with a basic hydrolyzing agent to yield a pH of greater than 7, such as about 8-14, about 11-13, or about 12-12.5. Any suitable base may be used to increase in pH, for example, sodium hydroxide, or other strong base, such as potassium hydroxide may be used. Temperature, time, and pH may be varied to achieve more efficient digestion.

This basic slurry may be optionally heated. When heated, the slurry may reach temperatures of from about 1-200° C., such as about 20-100° C., about 50-95° C., or about 90° C. When temperatures above 100° C., or the boiling point of the solution are used, an apparatus capable of withstanding pressures above atmospheric pressure may be employed. In some embodiments, depending on the type of algae, the type and concentration of acid used for hydrolysis, the outside temperature conditions, the permissible reaction time, and the conditions of the slurry, heating may be omitted. Heating may occur prior to, during, or after addition of a hydrolyzing agent.

In addition, the basic slurry may be optionally mixed either continuously or intermittently. Alternatively, a hydrolysis reaction vessel may be configured to mix the slurry by convection as the mixture is heated.

Basic hydrolysis may be permitted to take place for a suitable period of time depending on the temperature of the slurry and the concentration of the hydrolyzing agent. For example, the reaction may take place for up to 72 hours, such as from about 12-24 hours. If the slurry is heated, then hydrolysis may occur at a faster rate, such as from about 15-120 minutes, 30-90 minutes, or about 30 minutes.

During acid and base hydrolysis, chlorophyll may be hydrolyzed to the porphyrin head and phytol side chain, as well as complex polysaccharides are hydrolyzed to oligosaccharides or their respective monosaccharide component.

B. Biomass and Aqueous Phase Separation

Under the condition of elevated pH, the biomass may be separated from the aqueous solution. This separation is performed while the pH remains high to keep the lipids in their soap form so that they are more soluble in water, thereby remaining in the water phase. Any suitable separation technique may be used to separate the liquid (aqueous) phase form the biomass. For example, centrifugation, gravity sedimentation, filtration, or any other form of solid/liquid separation may be employed.

Once the separation is complete, the aqueous phase is kept separate and the remaining biomass may be optionally washed with water to help remove any residual soap molecules. This wash water may also be collected along with the original liquid phase. Once the biomass is washed it may be discarded or used in other bioproducts processes, such as solvent production as is described in U.S. Provisional Patent Application No. 61/552,317, the entirety of which is herein incorporated by reference.

The aqueous phase, which now contains the recovered lipids in soap form, Porphyrin salts, sugars, and any other soluble cellular components, may be processed further to derive bioplastics. Much of the hydrophobic or insoluble cellular components are potentially removed with the biomass, for example, pigments such as carotenoids.

C. Precipitate Formation

After the biomass is removed, the pH of the collected liquid may be neutralized/reduced to form a precipitate. This may be accomplished by the addition of an acid to the solution, such as at least one strong acid or mineral acid, for example, sulfuric, hydrochloric, phosphoric, or nitric acid. Addition of a suitable acid is performed until a green precipitate is formed. The green precipitate may contain, or may be, the Porphyrin heads as they are converted from their salt forms. It may also contain proteins and other cellular components that are coming out of solution.

The pH may be reduced to a pH of about 7 or less, such as about 4-6.9. This lower pH also converts the soap in the liquid to free fatty acids. As the precipitate forms the fatty acids associate with the solid phase and come out of solution. Once the precipitate has formed, the solid and liquid phases may be separated. Any suitable separation method may be employed, such as centrifugation, gravity sedimentation, filtration, or any other form of solid/liquid separation. The liquid phase may be taken for further processing. The collected solid phase may then be removed and further processed into other useful products, such as biodiesel, as described in U.S. Provisional Application No. 61/551,049, the entire disclosure of which is hereby incorporated by reference in its entirety. Optionally, the precipitate may be lyophilized or dried, which may result in nearly complete extraction of the lipids during extraction.

III. Bioplastic Production

A. Bacteria

Any suitable bacterial strain capable of producing bioplastics may be used. For example, the Escherichia coli strain described in U.S. patent application Ser. No. 12/907,572, filed Dec. 19, 2010, the entirety of which is herein incorporated by reference.

B. Growth Medium

The liquid/aqueous phase may be used directly as a medium for growth of bacteria capable of producing bioplastics or any other bioproducts. The liquid phase may be optionally augmented with other growth mediums and/or components, such as liquids, nutrients, minerals, and growth factors. The growth medium may contain at least 0.1% glycerol, such as at least 0.5% glycerol, or from 0.1 to about 20% glycerol, or from about 0.5 to about 15%. In addition to glycerol the liquid/aqueous phase may also contain other (undefined) simple sugars that the bioplastics-producing microbe can use as a carbon source. Furthermore, the liquid/aqueous medium is at an optimum salt/ion concentration which provides the ideal buffering capacity for the bacteria to grow and produce PHB. The liquid media also does not inhibit the effect of antibiotics or the inducer Isopropyl β-D-1-thiogalactopyranoside (IPTG), which are required for the maintenance of the pBHR68 plasmid and the start of PHB gene expression respectively. In some embodiments, the growth medium may be used alone or in combination with other growth mediums for fermenting any bacterial strain that requires a sugar source for growth.

C. Growth of Bacteria

The bacteria may be grown or fermented in the growth medium at a suitable temperature for a suitable period of time to maximize production of bioplastics. Fermentation may be undertaken in small or large fermenters in either a batch or continuous setup. Typically, the bacteria are grown at about 37° C. for a period of about 1 to 4 days, such as about 48 hours.

D. Purification

After fermentation, the bioplastics may be purified from the medium depending on the bacteria strain used. In some embodiments, the bacteria may be separated from the growth medium (which may be optionally or partially recycled) by a suitable separation method, such as filtration, centrifugation, etc.

Any suitable purification technique may be used. The PHB may be directly quantified using the NMR/GC method outlined in the Examples below. In such a method, bacterial cells may be subjected to bleach and chloroform. The bleach lyses open the cells, liberating the PHB into the chloroform phase. In embodiments using PHB secreting bacteria, the bacterial culture was treated with CaCl₂ to separate the secreted PHB from the non-secreted PHB.

### EXAMPLES

The following examples are illustrative only and are not intended to limit the disclosure in any way.

E. coli strain harboring the pBHR68 plasmid was cultured in culture medium derived from the algal strains associated with or without flocculants as follows:

| Sample | Algae Strain | Flocculent |
|---|---|---|
| 1 | Scenedesmus obliquus | Aluminum Sulfate |
| 2 | Scenedesmus obliquus | Modified potato starch |
| 3 | Scenedesmus obliquus | None |
| 4 | Logan Lagoons Algae | Modified corn starch |
| 5 (control) | None | None |
| 6 | Logan Lagoons Algae | Centrifuged |
| 7 | Logan Lagoons Algae | Aluminum Sulfate |
| 8 | Logan Lagoons Algae | Modified potato starch |

Ten sample culture mediums were derived by performing acid hydrolysis, base hydrolysis, biomass and aqueous phase separation, and pellet formation as described above to produce a liquid phase from the above feedstock materials. Once products were received, each sample had 100 mL centrifuged at 3500 rpm for 25 min. The supernatant was placed in a beaker and pH was adjusted to approximately pH 7 with NaOH. It should be noted that all samples had an initial pH of less than 3 before neutralization. These neutralized samples were then divided into separate flasks (100 ml of each sample in each flask). Each flask was autoclaved at 121° C. for 25 min.

The control flask consisted of 20 mL solution of 10 g YE+75 g glucose per L)+10 mL 10× M9+0.02 mL MgSO₄+70 mL H₂O.

To each sample flask was added 100 μL Amp50, 100 μL IPTG, 1 mL pBHR68 (non-secreting). The flasks were placed at 37° C. on a shaker table and bacterial growth (colony forming units CFU/mL) was measured at 0, 4, 8, 12, 24, and 48 hrs. After 48 hours samples were centrifuged at 3500 rpm for 25 min. The resulting pellet was then freeze dried for 48 hours. Freeze dried samples were then processed for NMR analysis. An NMR-GC correlation was used to determine the PHB concentration in each sample. See E. Linton, A. Rahman, S. Viamajala, R. C. Sims, C. D. Miller, Polyhydroxyalkanoate quantification in organic wastes and pure cultures using a single-step extraction and 1H NMR analysis, Water Science and Technology, Accepted Manuscript (2012).

The results of these samples are summarized below:

Medium for Growth
After neutralization of the aqueous phase from WLEP, it can be used as a suitable medium for bacterial growth.
While the dominate carbon source is expected to be glycerol, there could be other simple sugars in the media that aid in growth.
There are micronutrients (such as salts) in the aqueous phase that provide a suitable medium for bacterial growth.

Bacterial Growth and Viability
Bacterial growth was seen for all samples.
Bacterial growth (CFU/mL) was calculated for all samples. Samples grown in the aqueous phase from single strain algae (Scenedesmus obliquus) had higher CFU/mL on average than samples grown in Lagoon algae aqueous phase.

Bioplastic Production
Bacterial growth was seen in alum samples. However, no PHA production seen in these samples. This could mean that PHA being produced is below the detection limit of the NMR.
PHB was seen in single strain Scenedesmus obliquus flocculated with potato starch and processed with WLEP. From this it can be assumed that all other algae strains will act similarly.
PHB was seen in single strain Scenedesmus obliquus with traditional centrifugation and processed with WLEP
Bioplastic was seen in Logan Lagoon algae flocculated with corn starch and processed with WLEP (partially addresses the objectives outlined in overall Lagoon/combined patent).
Yields of bioplastic from processed single strain and mixed algae were similar (without replicates), however these yields were less than that seen in the control.

Laboratory Grade Glycerol
When compared to LB control, bioplastics-producing bacteria growing in M9-glycerol did not reach the same OD.
It was shown with NMR spectra that PHB can be produced using glycerol as the sole carbon source.

Determination of Glycerol Concentration in Aqueous Phase
From using a commercial kit (Biovision free glycerol assay kit), the aqueous phase was found to have 0.05 g/L concentration of glycerol.
In addition, there could be other simple sugars in the aqueous phase that still need to be analyzed. These simple sugars could have aided in the growth of bacteria.

The results are summarized in the following table (PHB yields were calculated using NMR/GC correlation):

| Sample/ | | PHB | |
|---|---|---|---|
| Flask | | peaks | Concentration |
| number | Description | present? | mg/mL |
| 1 | Alum only, Algae source: | No | |
| | Scenedesmus Obliquus | | |
| 2 | Potato starch only, Algae source: | Yes | 0.086 ± 0.032 |
| | Scenedesmus Obliquus | | |
| 3 | centrifuged, Algae source: | Yes | 0.089 ± 0.027 |
| | Scenedesmus Obliquus | | |
| 4 | Corn, Algae source: Logan | Yes | 0.084 ± 0.014 |
| | Lagoons | | |
| 5 | enhanced M9 media | Yes | 0.38 ± 0.05 |
| 6 | centrifuged, Algae source: Logan | Yes | 0.044 ± 0.014 |
| | Lagoons | | |
| 7 | Alum only, Algae source: Logan | No | |
| | Lagoons | | |
| 8 | Potato starch only, Algae source: | Yes | 0.070 ± 0.035 |
| | Logan Lagoons | | |

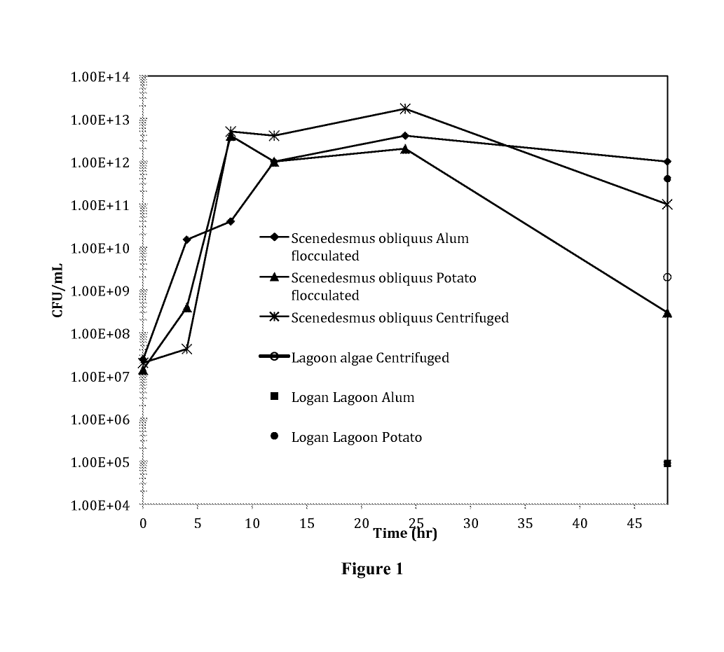

FIG. 1 illustrates CFU/mL for Samples 1-3 and 6-8.

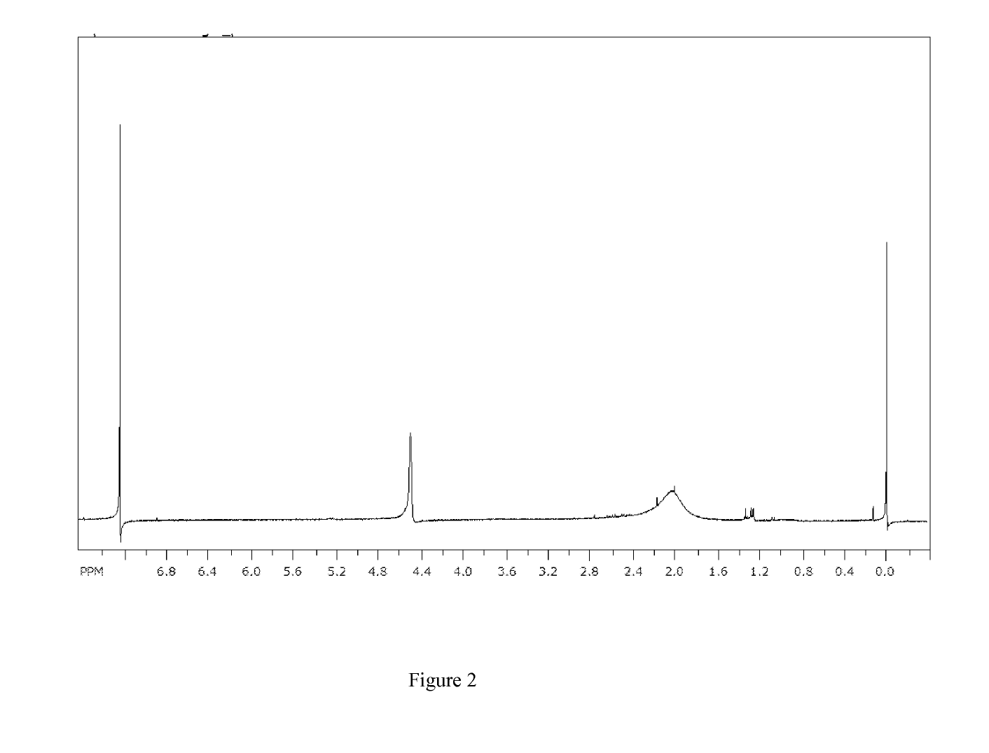

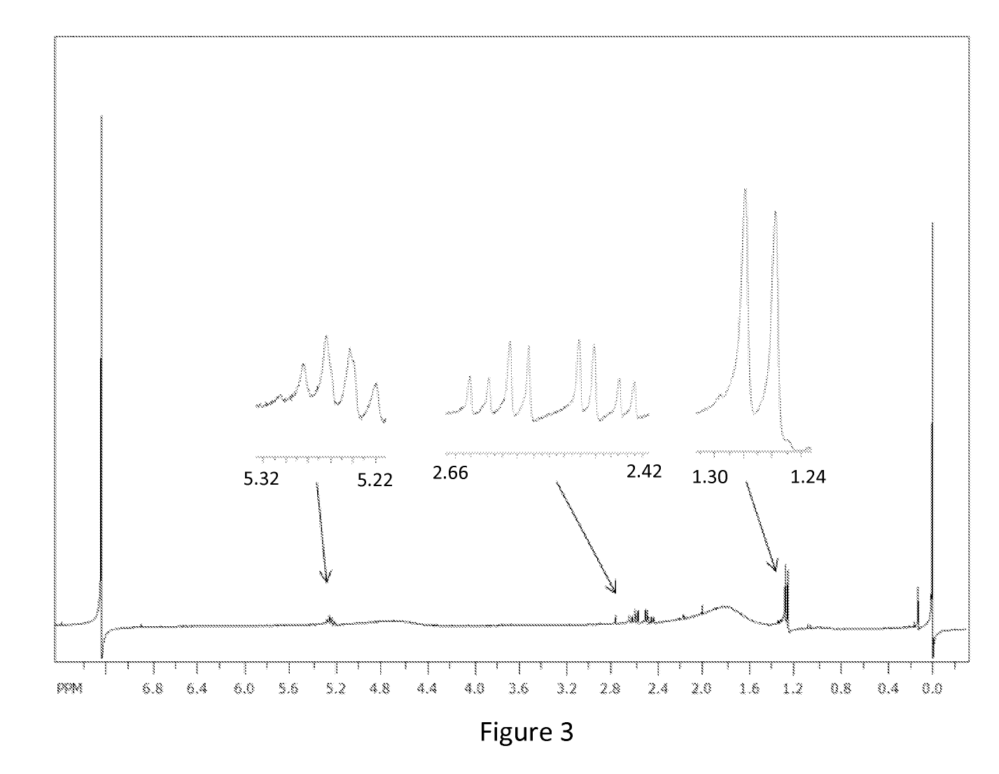

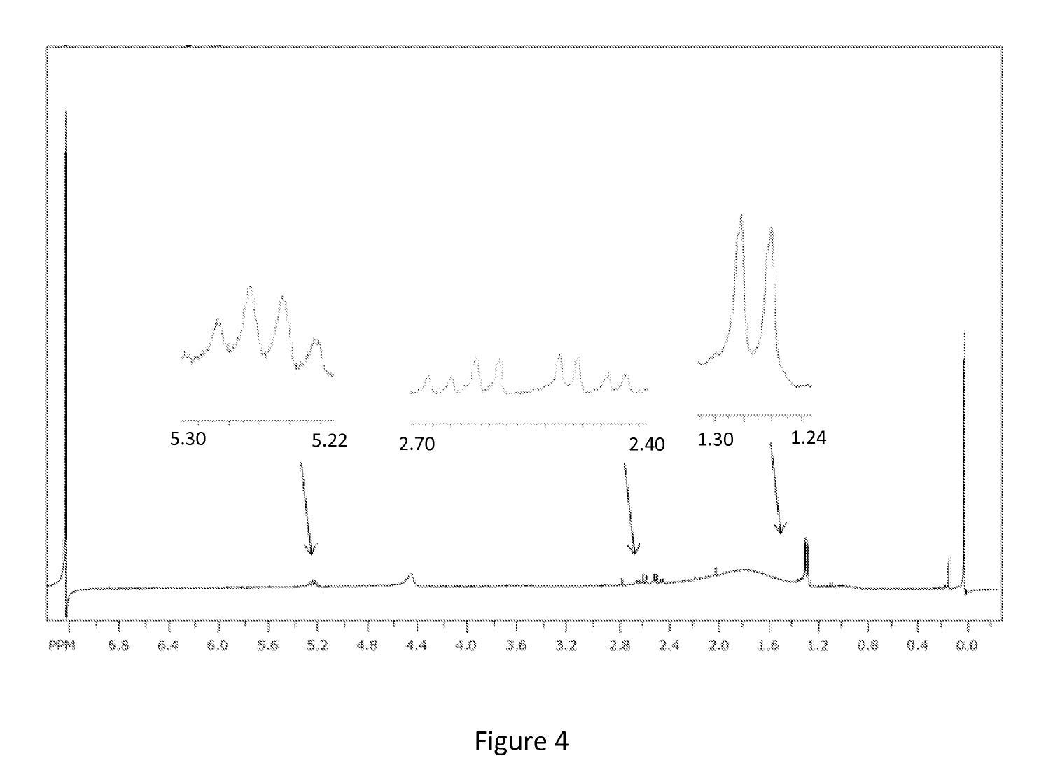

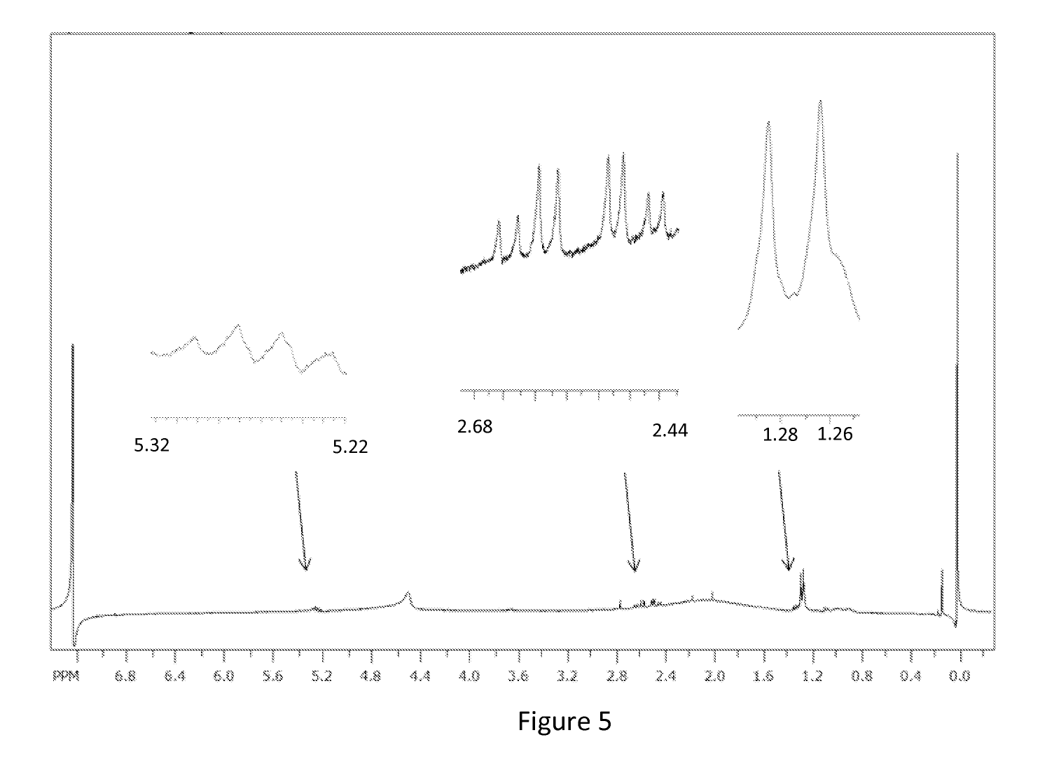

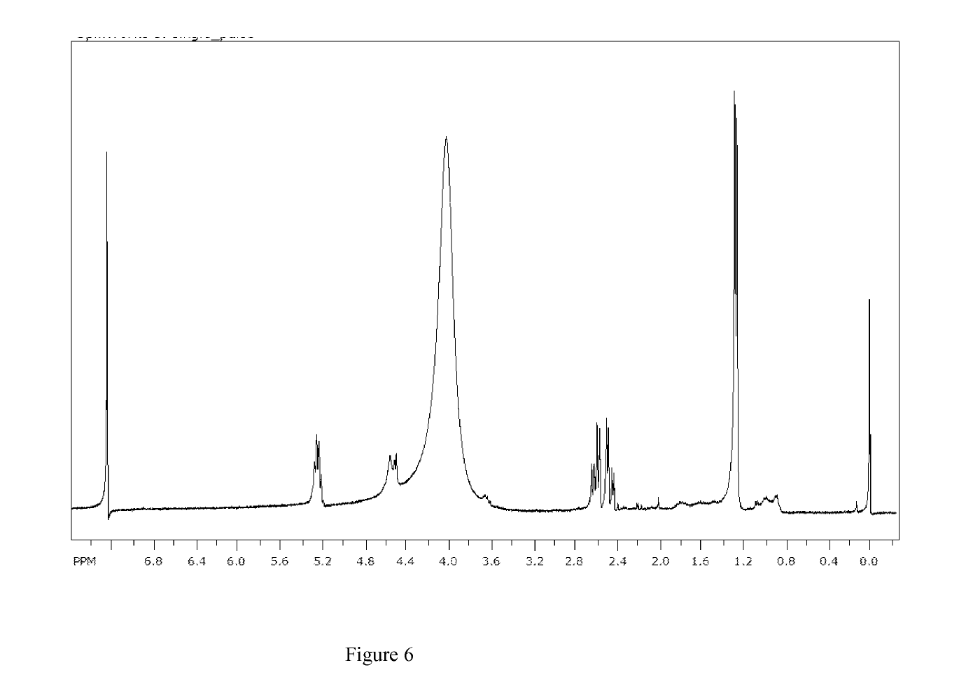

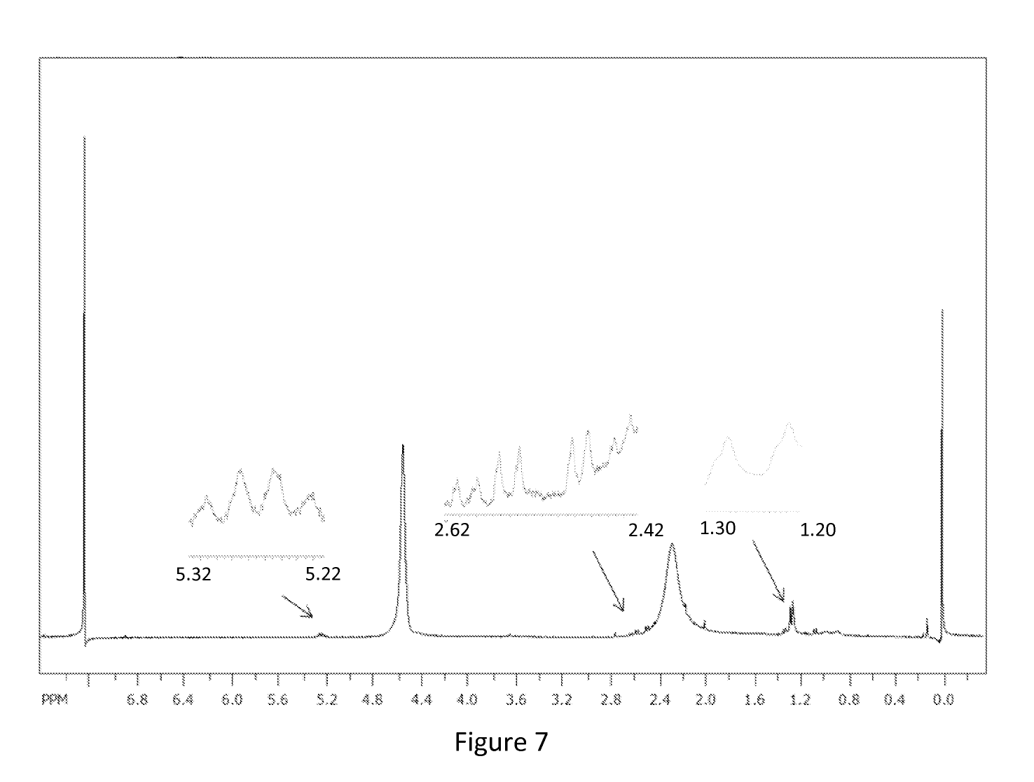

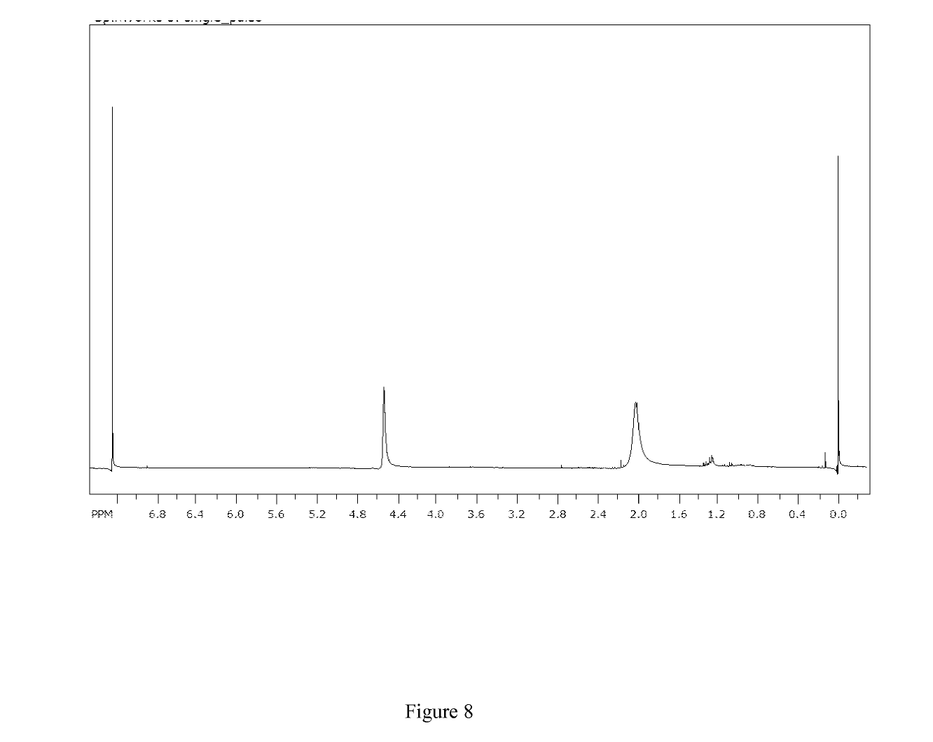

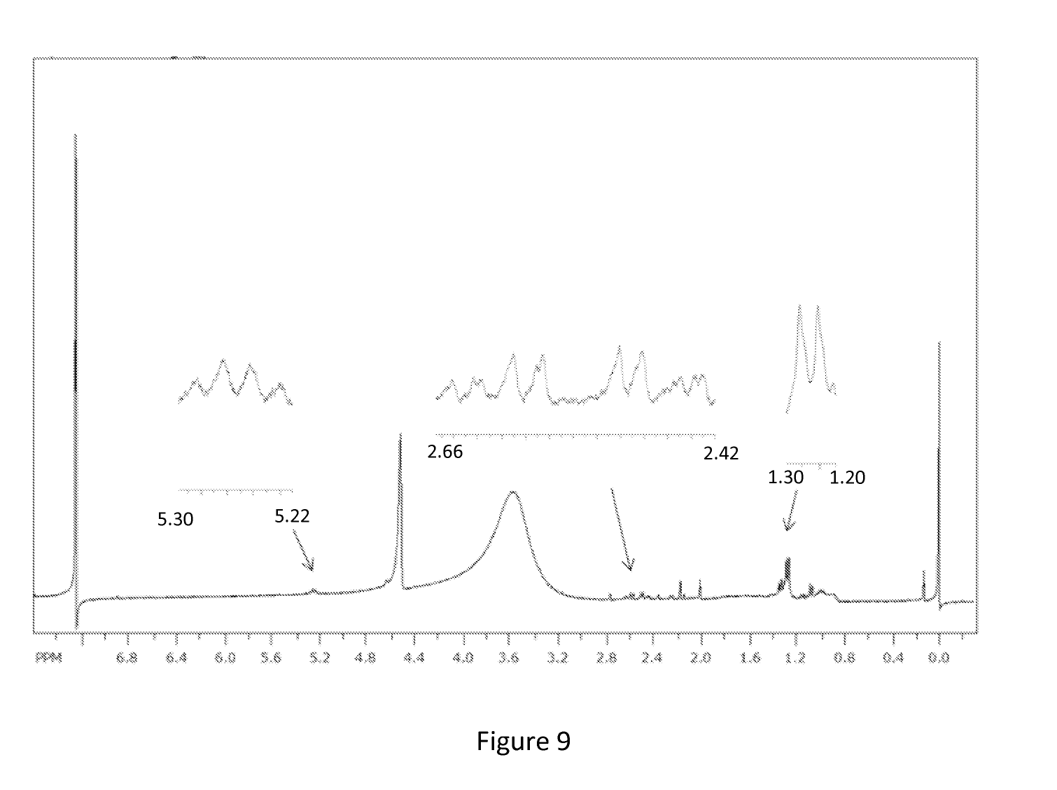

NMRs of Samples 1-8 are respectively illustrated in FIGS. 2-9.

Pure Glycerol Example:

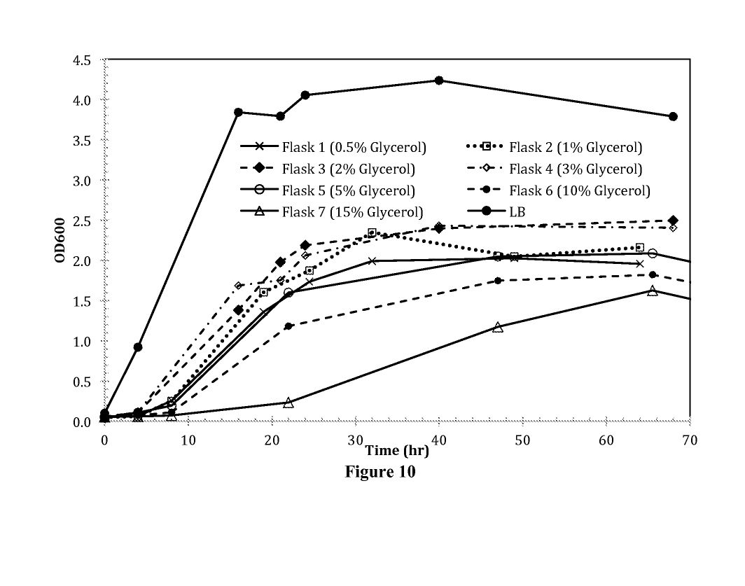

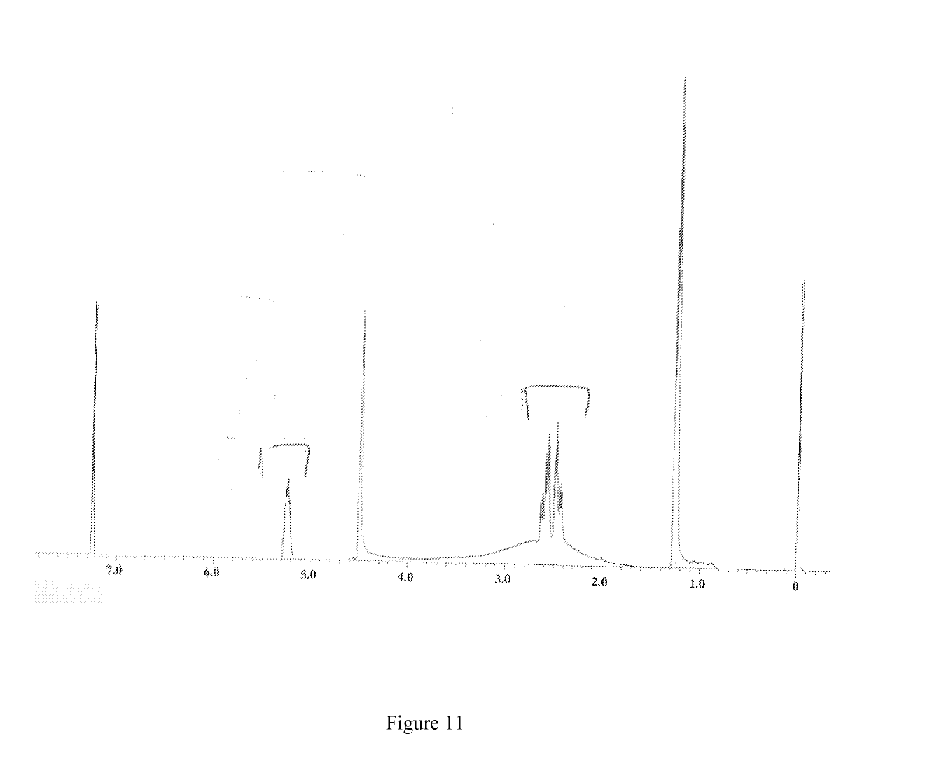

Bacterial strains harboring the plasmids 4MHT in pBHR68+pLG575 were grown in M9-glycerol media. The results are illustrated in FIGS. 10 and 11. FIG. 10 shows a graph of OD600 vs. time (hr) for different concentrations of glycerol in M9 media. FIG. 11 shows NMR spectra showing positive PHB peaks for secreting strain growing on glycerol as sole carbon source. From this example it is shown that PHB may be generated from laboratory grade glycerol. This example demonstrates growth of PHB producing strains on different concentrations of glycerol (0.5-15%).

Can see bioplastic produced from M9+glycerol. Demonstrates that PHB secreting strain of bacteria can be grown in ‘pure’ glycerol and also produce PHB.

It will be appreciated that various of the above-disclosed and other features and functions, or alternatives thereof, may be desirably combined into many other different systems or applications. Also, various presently unforeseen or unanticipated alternatives, modifications, variations or improvements therein may be subsequently made by those skilled in the art, and are also intended to be encompassed by the following claims.

What is claimed is:

1. A method of producing bioplastics from algae, the method comprising:
processing algae to yield an aqueous phase containing glycerol, and
fermenting the aqueous phase with a bioplastic-producing bacteria to yield bioplastics.

2. The method of claim 1, wherein processing algae comprises:
hydrolyzing a slurry comprising algae and water by adding an acidic hydrolyzing agent to yield an acidic slurry,
hydrolyzing the acidic slurry by adding a basic hydrolyzing agent to yield a basic slurry, and
separating the aqueous phase from biomass.

3. The method of claim 2, wherein the slurry has a solid content of about 4-25%.

4. The method of claim 2, wherein the acidic hydrolyzing agent is selected from the group consisting of a strong acid, a mineral acid, sulfuric acid, hydrochloric acid, phosphoric acid, and nitric acid.

5. The method of claim 2, wherein the acidic slurry has a pH of from about 1.5-4.

6. The method of claim 2, wherein the acidic slurry is heated to a temperature of from about 50-120° C.

7. The method of claim 2, wherein the basic hydrolyzing agent is selected from the group consisting of a strong base, sodium hydroxide, and potassium hydroxide.

8. The method of claim 2, wherein the basic slurry has a pH of from about 8-14.

9. The method of claim 2, wherein the basic slurry is heated to a temperature of from about 50-120° C.

10. The method of claim 1, wherein the bioplastic-producing bacteria are selected from the group consisting of Escherichia coli containing plasmid(s) for bioplastic production.

11. The method of claim 1, wherein the bioplastic produced is a polyhydroxyalkanoate (PHA) or a polyhydroxybutyrate (PHB).

12. The method of claim 1, further comprising purifying the bioplastic.

13. The method of claim 1, wherein the aqueous phase contains at least 0.05 g/L of glycerol.

14. The method of claim 1, further comprising harvesting the algae prior to processing the algae.

15. The method of claim 14, wherein:
harvesting the algae comprises mixing a modified starch with an aqueous solution containing the algae to be harvested and
forming flocs comprised of the modified starch and the algae.

16. A growth medium for growing bacteria, comprising an aqueous phase produced by hydrolyzing a slurry comprising algae and water by adding an acidic hydrolyzing agent to yield an acidic slurry,
hydrolyzing the acidic slurry by adding a basic hydrolyzing agent to yield a basic slurry, and
separating the aqueous phase from biomass.
